(19) United States
(12) Patent Application Publication
(10) Pub. No.: US 2021/0196467 A1
(43) Pub. Date: Jul. 1, 2021

(54) DISC PROSTHESIS FOR CERVICAL VERTEBRA HAVING ELASTICITY SIMILAR TO THAT OF BONE TISSUE

(71) Applicant: Soonchunhyang University Industry Academy Cooperation Foundation, Asan-si (KR)

(72) Inventor: soo bin Im, Bucheon-si (KR)

(73) Assignee: Soonchunhyang University Industry Academy Cooperation Foundation, Asan-si (KR)

(21) Appl. No.: 17/136,039

(22) Filed: Dec. 29, 2020

(30) Foreign Application Priority Data
Dec. 30, 2019 (KR) 10-2019-0177477

(51) Int. Cl. A61F 2/44; A61F 2/30

(52) U.S. Cl. CPC A61F 2/442; A61F 2/3094; A61F 2310/00023; A61F 2002/30578; A61F 2002/30985; A61F 2002/30574

(57) ABSTRACT

The present disclosure discloses a disc prosthesis for cervical vertebra having elasticity similar to that of bone tissue, and includes: a first support frame having a square shape; a second support frame formed to face the first support frame and spaced apart at a distance; and a mesh member integrally installed between the first support frame and the second support frame, wherein a first flange for supporting a fixing bolt is integrally formed on one side of the first support frame, and a second flange for supporting a fixing bolt is integrally formed on one side of the second support frame.

### CROSS-REFERENCE TO RELATED APPLICATIONS

This application claims priority to and the benefit of Korean Patent Application No. 10-2019-0177477 filed on Dec. 30, 2019, the disclosure of which is incorporated herein by reference in its entirety.

### TECHNICAL FIELD

The present disclosure relates to a disc prosthesis for a cervical vertebra, and more specifically, to a disc prosthesis for cervical vertebra having elasticity similar to that of bone tissue, which allows to be fixed to a strong part of bone through a strong coupling force using a material having elasticity similar to that of bone tissue.

### BACKGROUND

Discs that interconnect of spine bones from the neck, as people get older, undergo regressive changes to become less elastic, and a water content is reduced to generate disc wear or cracks, and thus it is common to lose a buffering function that is a main function, and when the changes progress further here, fibers will increase severely or a part of the disc will escape, and a bony spur will pop out and pain occurs in the shoulder, upper limb, and shoulder blades, etc. by pressing a nerve root located on the back side, and after that, as a distance between the spines gradually narrows, or the spine bones sink, spinal deformity may occur.

At this time, when the pain is not resolved by a conservative therapy, or progresses to nerve paralysis, surgery to remove a disc of cervical vertebra and a nerve compression site and insert a new prosthesis will be performed, and a commonly used prosthesis was a fusion material that caused fusion.

In related art, as shown in FIG. 1, a titanium cage was used as a prosthesis to support the cervical vertebra, but while a titanium material has good bone surface engraftment and does not reject the human body, it has very low elasticity, and thus, as shown in FIG. 1, there was a problem that a subsidence phenomenon in which the titanium material penetrates into the bone occurs.

In order to solve such a problem, a prosthesis formed of polyetheretherketon (PEEK) or a carbon composite polymer material was used, but such a material has little ability to engraft onto a surface of the bone tissue and is not attached to the bone, and thus there was a problem that the prosthesis was often detached from the bone.

In order to prevent the prosthesis from detaching, a tooth-shaped protrusion was made larger and sharper in upper and lower portions of the prosthesis, but such a protrusion has a very small area of contact with the bone at the initial stage, and an initial pressure applied to the bone is strong, and thus the subsidence phenomenon that the protrusion penetrates into the bone was promoted.

Therefore, in order to solve such a detaching problem, as shown in FIGS. 2A to 2C, a method of using a fixing bolt when fixing to the bone was introduced, and in order to prevent the fixing bolt from protruding forward, a method of fixing to a middle part of the bone (an inner part of the bone) was adopted when supporting and coupling to the disc prosthesis. This will go through a weak part of the bone. Since a cervical vertebra bone has a strong strength at a corner portion of edge and a weak structure on an inner surface thereof, a screw inserted inside the bone, not the edge, is not firmly coupled to the bone and rather breaks the bone by frequent movement of the neck.

In addition, there is a limit to a length in which the screw may be used in order to prevent the screw from touching nerves. There is no choice but to reduce the length of the screw, and as the length of the screw becomes shorter, the fixing becomes weaker.

In addition, the prosthesis has a hybrid-type body in which existing titanium is used for the front through which the screw pass and the back is formed of PEEK. The existing titanium that is filled inside has the elasticity of metal, and thus there was an adverse effect that the elasticity could not act in the front portion of the cervical vertebra, which requires the most elasticity.

Another problem with the method of fixing inside the cervical vertebra portion is that it is difficult to make a screw hole exactly at an intended place. The inside of the cervical vertebra into which the screw should penetrate has a concave shape. A puncher tool (e.g., awl or drill) is used in advance before inserting the screw, and a concave surface of the bone and the puncher tool form an acute angle. While the puncher tool enters, it makes contact with the bone surface at an acute angle and slides backward. The screw hole is formed behind the originally intended point, and when the screw is inserted, the prosthesis also moves backward. It becomes difficult to fix the prosthesis in the correct position, and when the posteriorly moved prosthesis compresses posterior nerves, serious neurological symptoms may occur.

Therefore, there has been a continuous demand for disc prosthesis for cervical vertebra, which is made of a material that may fix more effectively, has elasticity similar to that of bone, and adheres well to bones due to its excellent bone surface engraftment.

### PRIOR ART LITERATURE

### Patent Literature

(Patent Document 1) Korean Laid-open Patent Application No. KR10-1964862 (Mar. 27, 2019)
(Patent Document 2) Korean Laid-open Patent Application No. KR10-1484073 (Jan. 13, 2015)

### SUMMARY

### Technical Problem

An object of the present disclosure devised to solve the above-described problems is directed to providing a disc prosthesis for cervical vertebra that may have a strong coupling force and position a prosthesis in a correct position by fixing to a strong corner portion of bone of the cervical vertebra through a flange structure.

Another object of the present disclosure is directed to providing a disc prosthesis for cervical vertebra that has the elasticity of the entire prosthesis similar to that of the bone tissue by making the inside of the prosthesis so as to have a porous mesh structure by using a 3D-printed titanium material, and at the same time, may significantly improve bone surface engraftment so as to well couple to the bone, and may significantly reduce a subsidence phenomenon that penetrates into the bone.

### Technical Solution

To achieve the above-mentioned objectives, a disc prosthesis for cervical vertebra having elasticity similar to that of bone tissue according to the present disclosure includes: a first support frame having a square shape; a second support frame formed to face the first support frame and spaced apart at a distance; and a mesh member integrally installed between the first support frame and the second support frame.

A first flange for supporting a fixing bolt is integrally formed on one side of the first support frame, and a second flange for supporting a fixing bolt is integrally formed on one side of the second support frame.

The first flange is formed with a first coupling hole into which the fixing bolt is inserted, and the second flange is formed with a second coupling hole into which the fixing bolt is inserted.

The mesh member has a structure in which a plurality of star-shaped unit pieces are gathered to form a mesh structure, and has a structure capable of increasing the bondability with a living tissue while maintaining an elastic force.

The first flange and the second flange are positioned at a corner portion of edge of a cervical vertebra bone.

The fixing bolts inserted into the first coupling hole of the first flange and the second coupling hole of the second flange are inserted in a diagonal direction from the corner portion of edge of the cervical vertebra bone.

The disc prosthesis is molded by 3D-printing using a 3D-printed titanium material.

### Advantageous Effects

According to the present disclosure, there is an advantage that a strong coupling force can be obtained by fixing to a strong portion of bone through a flange structure. The flange completely prevents the prosthesis from slipping backward when creating a screw hole and performing a screw fastening operation.

According to the present disclosure, there is an effect of being able to effectively bio-bond to bone tissue through a porous structure or a mesh structure.

According to the present disclosure, there is an effect that the elasticity is similar to that of bone tissue by using a 3D-printed titanium material, it is possible to prevent the prosthesis from penetrating into the bone, and at the same time, it is possible to couple well to the bone by significantly improving bone surface engraftment.

### BRIEF DESCRIPTION OF THE DRAWINGS

FIG. 1 is a view showing a disc prosthesis for cervical vertebra according to the related art.

FIGS. 2a to 2c are views showing an installation state of the disc prosthesis for cervical vertebra according to the related art.

FIGS. 3A to 3C are schematic diagrams of a usage state of a disc prosthesis for cervical vertebra according to the present disclosure.

FIG. 4 is a front perspective view of the disc prosthesis for cervical vertebra according to the present disclosure.

FIG. 5 is a side perspective view of the disc prosthesis for cervical vertebra according to the present disclosure.

FIG. 6 is a plan perspective view of the disc prosthesis for cervical vertebra according to the present disclosure.

FIG. 7 is a perspective view showing a usage state of the disc prosthesis for cervical vertebra according to the present disclosure.

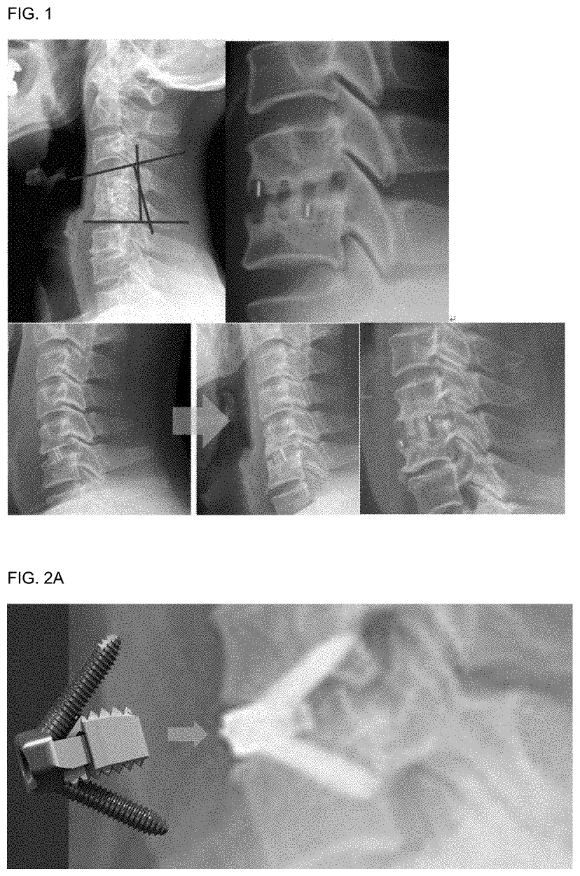

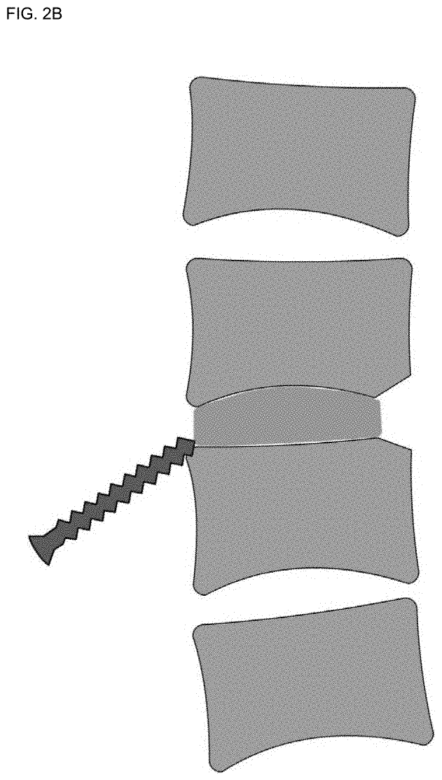

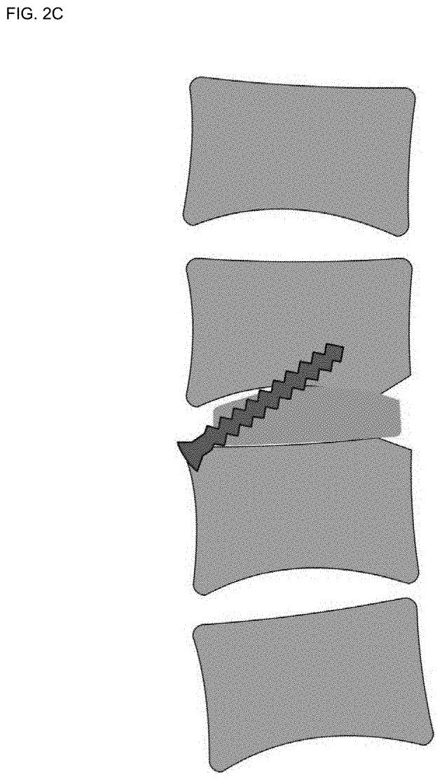

### DETAILED DESCRIPTION OF THE EMBODIMENTS

Hereinafter, a disc prosthesis for cervical vertebra according to the present disclosure will be described in detail with reference to the accompanying drawings.

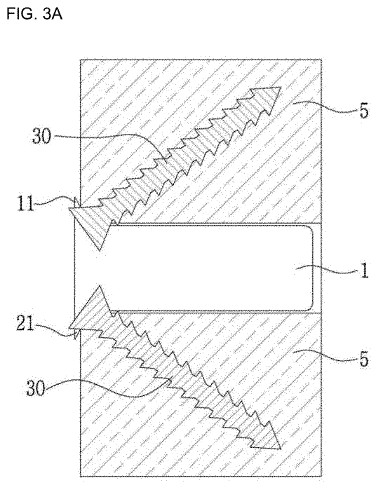

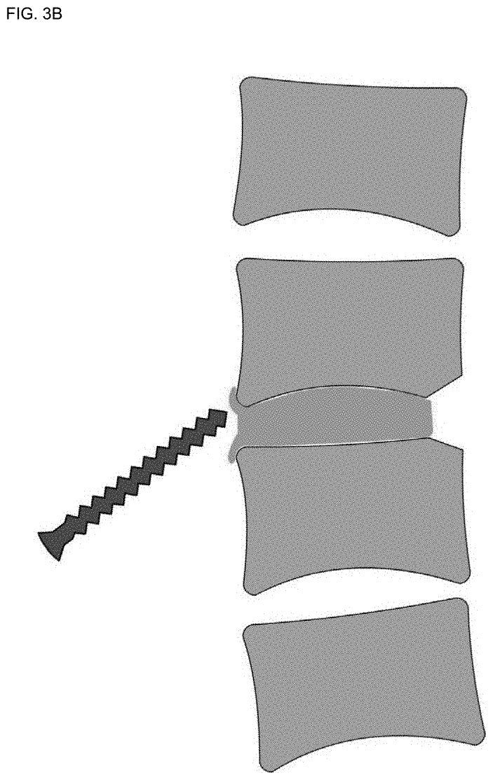

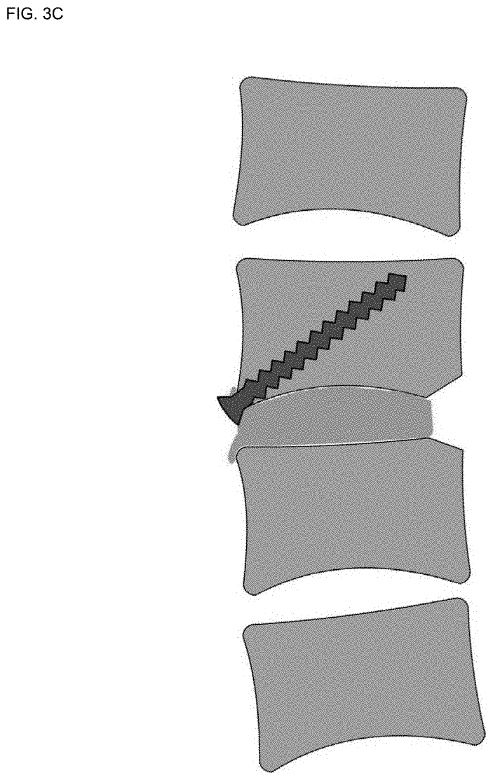

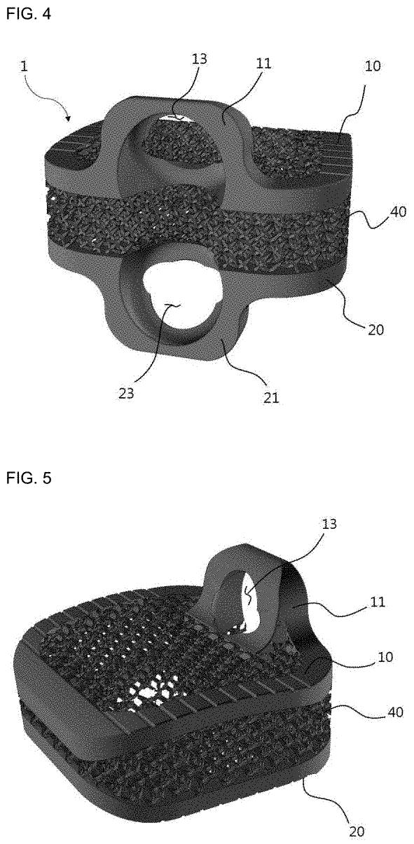

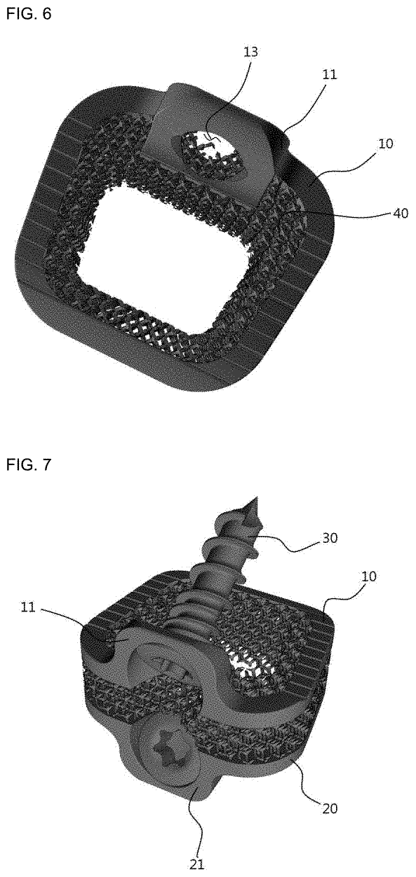

FIGS. 3A to 3C are schematic diagrams of a usage state of a disc prosthesis for cervical vertebra according to the present disclosure, FIG. 4 is a front perspective view of the disc prosthesis for cervical vertebra according to the present disclosure, FIG. 5 is a side perspective view of the disc prosthesis for cervical vertebra according to the present disclosure, and FIG. 6 is a plan perspective view of the disc prosthesis for cervical vertebra according to the present disclosure.

As shown in FIGS. 3A to 3C, a disc prosthesis for cervical vertebra 1 according to the present disclosure is installed between cervical vertebra bones 5, and is fixed by a fixing bolt 30 inserted through a first flange 11 and a second flange 21.

According to the present disclosure, the first flange 11 and the second flange 21 are positioned at a corner portion of edge of the cervical vertebra bone 5, and the fixing bolt 30 inserted through the respective flanges may travel diagonally from the corner portion, which is the strongest portion of the cervical vertebra bone 5. Since the fixing bolt 30 is inserted as described above, its length may be increased to use, and accordingly, a coupling force between the disc prosthesis 1 and the cervical vertebra bone 5 may be significantly increased.

The disc prosthesis for cervical vertebra 1 according to the present disclosure may be manufactured by 3D-printing using a 3D-printed titanium material. Accordingly, elasticity is similar to that of bone tissue so that it does not penetrate into the bone, and at the same time, it is possible to obtain an effect of being able to couple well to the bone by significantly improving bone surface engraftment.

As shown in FIGS. 4 to 6, the disc prosthesis for cervical vertebra 1 according to the present disclosure includes a first support frame 10 having a rectangular shape, a second support frame 20 formed to be separated at a distance so as to face the first support frame 10, and a mesh member 40 integrally installed between the first support frame 10 and the second support frame 20.

The first flange 11 for supporting the fixing bolt 30 is integrally formed on one side of the first support frame 10, and the second flange 21 for supporting another fixing bolt 30 is also integrally formed on one side of the second support frame 20. In addition, a first coupling hole 13 into which the fixing bolt 30 is inserted is formed in the first flange 11, and a second coupling hole 23 into which another fixing bolt 30 is inserted is formed in the second flange 21.

The first support frame 10, the mesh member 40, and the second support frame 20 are all integrally formed and connected to each other.

The mesh member 40 has a structure in which a plurality of star-shaped unit pieces are gathered to form a mesh structure, and has a structure capable of increasing the bondability with a living tissue while maintaining an elastic force.

Since the fixing force may be sufficiently increased by using the screw and the flange of the disk prosthesis, a structure having a sharp teeth shape that existed to increase a frictional force on upper and lower surfaces has been significantly reduced. The mesh structure as described above may increase a contact area between the bone tissue and the prosthesis at an initial stage and may disperse pressure applied to the bone tissue at the initial stage, thereby further reducing a subsidence phenomenon.

FIG. 7 is a perspective view showing a usage state of the disc prosthesis for cervical vertebra according to the present disclosure.

As shown in FIG. 7, a pair of fixing bolts 30 may be inserted into the cervical vertebra bone 5 through the first coupling hole 13 of the first flange 11 and the second coupling hole 23 of the second flange 21.

FIG. 8 shows the usage state for this. Referring to FIG. 8, the fixing bolt 30 is inserted in a diagonal direction from the corner portion of edge of the cervical vertebra bone 5, so that a long screw may be used to significantly improve the coupling force thereof without being in contact with a nerve portion positioned at rear of the cervical vertebra bone 5.

The present disclosure may function as described above because each flange may be positioned at the corner portion of edge of the cervical vertebra bone 5 by providing the first flange 11 and the second flange 21.

Those skilled in the art to which the present disclosure with the above contents pertains can understand that the present disclosure can be implemented in other specific forms without changing the technical spirit or essential features of the present disclosure. Therefore, the embodiments described above should be understood as being exemplary in all respects and not limiting.

### DESCRIPTION OF REFERENCE NUMERALS

| | |
|---|---|
| 1: | Disc prosthesis |
| 5: | Cervical vertebra bone |
| 10: | First support frame |
| 11: | First flange |
| 13: | First coupling hole |
| 20: | Second support frame |
| 21: | Second flange |
| 23: | Second coupling hole |
| 30: | Fixing bolt |
| 40: | Mesh member |

What is claimed is:

1. A disc prosthesis for cervical vertebra comprising:
a first support frame having a square shape;
a second support frame formed to face the first support frame and spaced apart at a distance; and
a mesh member integrally installed between the first support frame and the second support frame,
wherein a first flange for supporting a fixing bolt is integrally formed on one side of the first support frame, and a second flange for supporting a fixing bolt is integrally formed on one side of the second support frame.

2. The disc prosthesis for cervical vertebra of claim 1, wherein the first flange is formed with a first coupling hole into which the fixing bolt is inserted, and the second flange is formed with a second coupling hole into which the fixing bolt is inserted.

3. The disc prosthesis for cervical vertebra of claim 1, wherein the mesh member has a structure in which a plurality of star-shaped unit pieces are gathered to form a mesh structure, and has a structure capable of increasing the bondability with a living tissue while maintaining an elastic force.

4. The disc prosthesis for cervical vertebra of claim 1, wherein the first flange and the second flange are positioned at a corner portion of edge of a cervical vertebra bone.

5. The disc prosthesis for cervical vertebra of claim 4, wherein the fixing bolts inserted into the first coupling hole of the first flange and the second coupling hole of the second flange are inserted in a diagonal direction from the corner portion of edge of the cervical vertebra bone.

6. The disc prosthesis for cervical vertebra of claim 1, wherein the disc prosthesis is molded by 3D-printing using a 3D-printed titanium material.
